(19) United States
(12) Patent Application Publication
(10) Pub. No.: US 2026/0253686 A1
(43) Pub. Date: Aug. 27, 2026

(54) COMPUTATIONAL COMPLIANCE VERIFICATION OF VASCULAR ACCESS MANAGEMENT

(71) Applicant: Becton, Dickinson and Company, Franklin Lakes, NJ (US)

(72) Inventor: Amir Tahmasebi Maraghoosh, Newton, MA (US)

(21) Appl. No.: 19/063,708

(22) Filed: Feb. 26, 2025

(51) Int. Cl. G16H 10/60; G16H 30/20

(52) U.S. Cl. CPC G16H 10/60; G16H 30/20

(57) ABSTRACT

Systems and methods are provided for machine learning and real-time feedback technology to provide technical improvements to a clinical system. For example, the system may determine a monitored action being performed by a clinician and compare the action to a clinical step in a clinical procedure. Based on the comparison, the system may provide feedback in the form of a completion indicator of the individual clinical steps or the clinical procedure overall.

### BACKGROUND

Image recognition, in the context of machine learning, is a computational process that trains machine learning models to interpret and understand the visual world. The machine learning models can identify and categorize images, based on patterns and objects within them. By converting images into numerical or symbolic information, image recognition can convert real world objects into computationally understandable objects.

In some examples, an image is received as a digital photo or video frame and then processed to enhance the image and remove noise. This can include adjusting brightness, contrast, and other parameters to standardize the input. The processed image is then analyzed using machine learning. Features are extracted, which can be patterns, colors, textures, or shapes of the image. These features are then provided as input to the trained machine learning model to classify the image. The output is a prediction of what the image and features represent based on the training process, like a person or physical object.

### BRIEF DESCRIPTION OF THE DRAWINGS

The present disclosure, in accordance with one or more various examples, is described in detail with reference to the following figures. The figures are provided for purposes of illustration only and merely depict typical, non-limiting aspects of such examples.

FIG. 1 illustrates a compute component in communication with an image sensor, classification model engine, and feedback device, in accordance with some examples of the disclosure.

FIG. 2 illustrates an environment that comprises a sensor and feedback device, in accordance with some examples of the disclosure.

FIG. 3 illustrates an environment that comprises a sensor, in accordance with some examples of the disclosure.

FIG. 4 illustrates an environment that comprises a sensor, in accordance with some examples of the disclosure.

FIG. 5 illustrates an environment that comprises a sensor, in accordance with some examples of the disclosure.

FIG. 6 illustrates feedback associated with the image detection, in accordance with some examples of the disclosure.

FIG. 7 illustrates feedback associated with the image detection, in accordance with some examples of the disclosure.

FIG. 8 illustrates feedback associated with the image detection, in accordance with some examples of the disclosure.

FIG. 9 illustrates feedback associated with the image detection, in accordance with some examples of the disclosure.

FIG. 10 is a computing component that may be used to implement examples of the disclosed technology.

FIG. 11 depicts a block diagram of an example computer system in which various examples of the disclosed technology described herein may be implemented.

The figures are not exhaustive and do not limit the present disclosure to the precise form disclosed.

### DETAILED DESCRIPTION

In traditional image recognition systems, models may generate a prediction that the image includes a person or physical object. The output from the model is often a binary response. This may include “yes” or “true” as to whether the person or object is present in the image.

Examples of the disclosed technology (e.g., system, method, computer readable media, and related functionalities) are directed to machine learning and real-time feedback mechanisms that provide technical improvements to a clinical system. For example, a system may comprise cameras, microphones, or other types of sensors to monitor actions performed by a clinician. Based on the actions captured by these sensors, the system may determine that the actions are associated with a clinical procedure, correlate the action to a clinical step in such a clinical procedure, and generate feedback associated with the actions performed.

In this context, a “clinical procedure” may correspond to operations or practices (e.g., “clinical steps”) performed by a clinician to address a patient's medical needs. The clinical procedure may be performed in the presence of a patient or during a training process to help the clinician learn/practice the clinical steps. The clinical steps may be defined by national or local agencies or organizations and stored in a data store of the system. In some examples, clinical steps may be defined as rules that relate to the clinical procedure. The rules may include information regarding a clinical tool used, operations taken to orient the tool, a time duration of each operation, and other characteristics of the actions. As an illustrative example, the clinical procedure may involve vascular access management activities and the clinical steps of the clinical procedure may include, for example, a hub scrub, dressing placement and replacement, and other vascular access-related activities. In some examples, the vascular care relating to the clinical procedure may involve medication administration, constructing connectors or other devices, line care, catheter placement, and other forms of vascular access management. The rules relating to the clinical steps may define thresholds/ranges that are satisfied to complete the clinical step.

Examples of the disclosed technology may receive an indication of the clinical procedure from the national or local agencies or organizations. The indication may identify that information is stored in an accessible data store and available for access. Rules corresponding to the clinical steps of the clinical procedure may be determined from the information as well. Additional detail regarding the indication of the clinical procedure and determination of the clinical step as rules in the system are provided throughout the disclosure.

The system may also receive a digital file representative of a monitored action in a clinical setting. The digital file may be recorded using output from a sensor (e.g., cameras, microphones, etc.). The recording may represent an action or set of actions that is being performed by a clinician in the clinical setting. For example, the digital file may record at least one image of an action (e.g., twisting a syringe, moving a disinfectant wipe, etc.) or an object (e.g., an image of a clinician's hand, a syringe, a disinfectant wipe, etc.). The digital file may comprise a single image or set of images of the object/action.

The system can process the digital file using one or more machine learning models. The machine learning models may be trained to generate different inferences or may be implemented as a single machine learning model that performs multiple functions. For example, a first machine learning model (e.g., a foundational model) may detect patterns and objects in digital files. The first machine learning model may be fine-tuned to relate the patterns/objects in the digital file to actions/objects for clinical steps. A second machine learning model (e.g., clinical procedure machine learning model) may be trained to determine whether the clinical steps are conducted to a clinically appropriate level, as further discussed herein. In other examples, a single machine learning model may be trained to detect patterns/objects in the digital file, relate the patterns/objects to clinical steps, and determine whether the clinical steps are conducted to a clinically appropriate level, without diverting from the essence of the disclosure.

The disclosed technology may implement the machine learning models using a training phase and an inference phase for each model. For example, during a training phase and before the first machine learning model has been operationalized in a production environment, the first machine learning model may learn associations of the patterns and objects in digital files. The first machine learning model may be fine-tuned to relate the detected patterns/objects to the rules defining the clinical steps of a clinical procedure (e.g., which actions are performed). During an inference phase, when the first machine learning model is operational/in use, the digital file (corresponding to the recording of the actions) may be provided as input to the trained first machine learning model. The output of the trained first machine learning model may be provided to a second model (e.g., the clinical procedure machine learning model) to determine whether the rules corresponding to the clinical steps are satisfied. In other words, when two models are implemented, the first model can detect the patterns and objects in digital files and the second model can identify whether the objects/action/clinical steps are complete in accordance with the rules.

Clinical steps may be defined by a set of rules. Such rules may comprise thresholds or ranges for identifying a “clinically appropriate level” for each clinical step. The clinically appropriate level of the clinical step may comprise a comparison of the action identified in the digital file with the definition of the clinical step in the guidance from national or local agencies or organizations. For example, the clinical steps may be defined by rules on which clinical tool to use, operations taken to orient the tool, a time duration of each operation, and other characteristics of the actions. The completion of the rules may depend on completing the clinical procedure accurately and in-line with the guidance. The system may compare the detected action in the digital file with the set of rules of the clinical step to determine whether the threshold/range of each rule is met or not met. As an illustrative example, a clinical procedure may require performing a first clinical step for fifteen seconds followed by a second clinical step of positioning a tool at a 45-degree angle. The system can detect the actions in the digital file, including the duration of actions and orientation of the tools/objects, and determine whether the actions are detected for the appropriate duration, orientation, and angle.

Feedback may be provided in response to the comparison, including a set of values with a completion indicator. For example, the disclosed technology may identify when an activity is initiated (e.g., as a clinical step), which objects/tools are detected and connected to each other, whether objects/tools identified in the digital file are being used correctly, whether the action is conducted for a duration of time to a clinically appropriate level (e.g., to completion of the clinical step), a length of time that the action was performed or the object/tool was used, and other feedback discussed throughout the disclosure. The system may provide text, images, sound, or other feedback as a set of values related to these clinical steps and the completion of these clinical steps. The feedback may also comprise a completion indicator that illustrates/provides whether the action is performed at the clinically appropriate level to satisfy the rules of the clinical step in the clinical procedure. Using the feedback, the clinician can adjust the actions being performed to more closely align with the clinical step.

In some examples, the clinical steps may be used to generate feedback about completion of the overall clinical procedure. For example, the system may determine that set of rules for the clinical step are satisfied to the clinically appropriate level and also compare the number/percentage of completed clinical steps with the overall clinical procedure. The system may provide text, images, sound, or other feedback as a set of values related to the clinical procedure. The feedback may also comprise a completion indicator that illustrates/provides whether the clinical steps of the clinical procedure are satisfied to the clinically appropriate level.

The feedback may be provided through various means. For example, the feedback may be provided visually to a user interface (e.g., dashboard), through haptic feedback (e.g., buzzing or vibrating), through audio/visual feedback, or other feedback means. The timing of the feedback may assist with a clinical training process and allow the user to make real-time adjustments to actions in response to the feedback. In some examples, the feedback may identify whether the threshold/range is reached using a completion score (e.g., a numerical value like 100%) or a completion indicator (e.g., highlighting a value green/bold/underline, providing haptic feedback of reaching the threshold/range, activating/deactivating a clinical workflow, providing a numerical value like a length of time that the action was performed, etc.). Additional thresholds/ranges may be implemented as well (e.g., 50% to a clinically appropriate level is yellow/italic feedback, etc.).

Technical improvements are provided throughout the disclosure. For example, the described technology can improve machine learning models by incorporating a foundational model to detect objects in digital files and fine-tuning the model to ultimately detect the completion of clinical steps in a clinical procedure. This can allow the model used in the system to be trained faster than models used in traditional systems. As such, the described functionality is more efficient in image detection and generating real-time feedback for the clinician. There may also be fewer system resources dedicated to model training.

Additionally, the incorporation of sensors with real-time feedback can reduce the amount of time that is required to train clinicians to perform the clinical procedure. The reduction in training time can lead to a reduction in the size of the memory storage that is needed to store these images. Clinicians can be trained faster with more accurate and up-to-date feedback, which is an additional benefit of the described technology.

Another improvement accrues to the safe operation of the devices within the clinical environment. There are different errors that can occur at a device that may be due to actions that are detectable using one of more of the features described. In response to detecting an action (or omission of an action) that might cause such an error, a device can be adjusted to confirm or otherwise mitigate an adverse event.

Another improvement may be to avoid degrading clinical devices due to overuse or overcleaning. Medical connectors, for example, are designed for certain duration of use and being subjected to certain cleaning procedures. Deviations from the time of use or cleaning processes can affect the reliability of the device. Accordingly, the action detected by the features described can prolong the time the device is within “safe” operating limits. Furthermore, resources needed to clean or otherwise utilize the device (e.g., lubricant, gloves, gels, etc.) can be optimized to a level that meets the safe operational characteristics of the device and avoids over utilization that could impact safety or functionality of the device. This can provide additional environmental benefits by reducing the amount of material used in the clinical setting.

Another improvement specific to the clinical setting is related to automatic documentation of clinical actions. Clinical practice guidelines emphasize documenting activities related to patient care to ensure traceability should any questions arise about the care provided. There are some actions though that are not reportable directly by devices in the current clinical care settings such as vascular care actions. In today's systems, either these events are not reported. Alternatively, these events may be inferred based on other clinical actions that may not reliably indicate the intended action took place (e.g., inventory decrementing for a cleaning device may indicate someone intends to clean but does not prove the cleaning was performed or performed at the right level on the right device). Alternatively, the events may require a clinician to remember to return to the record keeping system and make an entry, but this can be time consuming and error prone. The features described can be used to automatically provide indication to a patient's electronic medical record that these events actually occurred. And since the system may have access to patient information and time information, a reliable record may be submitted with minimal occurrence of human data entry error.

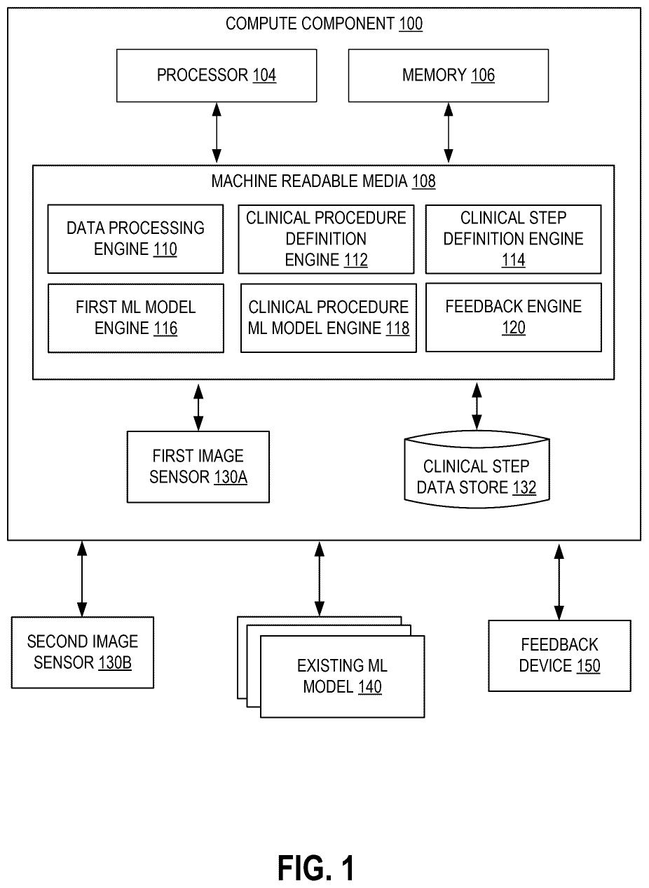

FIG. 1 illustrates a compute component in communication with an image sensor, classification model engine, and feedback device, in accordance with some examples of the disclosure. In this example, the clinical environment comprises compute component 100 that may be a server computer, a controller, or any other similar computing component capable of processing and transmitting data via network communications to the other devices. In the example of FIG. 1, compute component 100 comprises processor 104, memory 106, machine readable media 108, first image sensor 130A, and clinical step data store 132. Compute component 100 is communicable with (e.g., via wired or wireless communication connection) second image sensor 130B, second machine learning model engine 140, and feedback device 150.

Processor 104 may be one or more central processing units (CPUs), semiconductor-based microprocessors, and/or other hardware devices suitable for retrieval and execution of instructions stored in machine-readable storage medium 108. Processor 104 may fetch, decode, and execute instructions to control processes or operations for generating computational compliance verification of vascular access management. As an alternative or in addition to retrieving and executing instructions, processor 104 may include one or more electronic circuits that include electronic components for performing the functionality of one or more instructions, such as a field programmable gate array (FPGA), application specific integrated circuit (ASIC), or other electronic circuits.

Memory 106 may comprise random-access memory (RAM) or other dynamic memory for storing information and instructions to be executed by processor 104. Memory 106 might also be used for storing temporary variables or other intermediate information during execution of instructions to be executed by processor 104. Memory 106 may also comprise a read only memory (“ROM”) or other static storage device coupled to a bus for storing static information and instructions for processor 104.

Machine readable media 108 may comprise one or more interfaces, circuits, and modules for implementing the functionality discussed herein. Machine readable media 108 may carry one or more sequences of one or more instructions that can be executed by processor 104. Thus, machine-readable storage medium 108 may be, for example, Random Access Memory (RAM), non-volatile RAM (NVRAM), an Electrically Erasable Programmable Read-Only Memory (EEPROM), a storage device, an optical disc, and the like. In some examples, machine-readable storage medium 108 may be a non-transitory storage medium, where the term “non-transitory” does not encompass transitory propagating signals. Such instructions embodied on machine readable media 108 may enable compute component 100 to perform features or functions of the disclosed technology as discussed herein. For example, the interfaces, circuits, and modules of machine readable media 108 may comprise, for example, data processing engine 110, clinical procedure definition engine 112, clinical step definition engine 114, first machine learning model engine 116, clinical procedure machine learning model engine 118, and feedback engine 120. Any number or combination of these components may be included with compute component 100 or may be implemented remotely from compute component without diverting from the essence of the disclosure.

Data processing engine 110 is configured to receive a digital file. The digital file may be generated by image sensor 130 (illustrated as first image device 130A and second image device 130B). For example, image sensor 130 may comprise a camera, microphones, or other data generation device. Image sensor 130 may be incorporated with compute component 100 as a local image sensor (illustrated as first image sensor 130A) or may be located remotely from compute component 100 as a remote image sensor (illustrated as second image sensor 130B). Image sensor 130 may generate a digital file of a monitored action in a clinical setting. The digital file may correspond to a recording of an action or set of actions that is being performed by a clinician in the clinical setting.

The digital file may comprise time series data recorded by image sensor 130. The time series data may correspond to actions performed by a clinician in a clinical environment with respect to time, and the actions may be performed for a time duration. The digital file may identify an object (e.g., an image of a clinician's hand, a syringe, a disinfectant wipe, etc.) or an action (e.g., twisting a syringe, moving a disinfectant wipe, etc.) that is detectable in a single image or set of images.

The digital file may be received by data processing engine 110 from image sensor 130 through a data collection process. For example, the digital file may be transmitted from image sensor 130 to compute component 100 via a management process located at image sensor 130. The management process may transmit the digital file via an Application Programming Interface (API). In other examples, data processing engine 110 pulls the digital file from image sensor 130 via a wired or wireless connection.

Data processing engine 110 is also configured to pre-process the data. The pre-processing may clean the received data from image sensor 130. The pre-processing may remove noise, handle missing values, standardize formatting, and/or the like.

Clinical procedure definition engine 112 is configured to receive an indication of a clinical procedure that is defined by national or local agencies or organizations. For example, clinical procedure definition engine 112 may access a shared database of clinical procedures via a network and download a list of clinical procedures to a local data store, like clinical step data store 132. The list may comprise a description/name of the clinical procedure, clinical steps, and other relevant information for performing the clinical procedure.

As will be described in greater detail below, the clinical procedures may be defined by a set of clinical steps that need to be performed by the clinician. The clinical steps defined by the clinical procedure may also be accessed and downloaded to a local data store, like clinical step data store 132.

Clinical step definition engine 114 is configured to determine rules that satisfy a clinically appropriate level of each clinical step (e.g., as thresholds, ranges, scores, or other values). For example, in response to the guidance from the national/local agencies being stored in clinical step data store 132, as discussed with clinical procedure definition engine 112, the clinical procedure can be translated to clinical steps defined by the set of rules. Each clinical step can utilize objects/tools that are to be present and a set of actions to be performed in order to satisfy the rule and complete the clinical step.

Various rules may be defined in accordance with the guidance. For example, a rule for a clinical step may be associated with an object/tool, including a syringe, needle-free connector (NFC), alcohol swab, clinician's hands, patient, and other object that may be detected in a clinical environment. In some examples, the rule for a clinical step may be associated with an action using the object/tool, including holding the syringe/NFC/swab, scrubbing the NFC/hands, rotating the syringe, and so on.

As an illustrative example, a rule may define a rotation of an object/tool as part of a clinical step (e.g., twisting a syringe, etc.). The rule may include, for example, a threshold time/duration to perform the action (e.g., start, middle, and end), a threshold value in rotating the tool by the clinician, a threshold change in the angle of the tool during and after the action, and other information relating to the action. In another example, a rule may define a presence of an object/tool as part of a clinical step (e.g., a clinician's hand, a syringe, a disinfectant wipe, etc.). The rule may include, for example, a threshold time/duration to interact with the object (e.g., 15 seconds to wash hands at the start and end of the action, swabbing the NFC access surface for five seconds with 70% isopropyl alcohol, allowing the NFC to dry, etc.), a threshold value in the rotation/orientation of the tool by the clinician or patient, branding information that is associated with the object, and other information relating to the detected object. While these examples are provided for illustrative purposes, they should not be limiting to the disclosure herein.

The rules may be grouped into categories. For example, the categories of rules may be grouped by object/tool or threshold/ranges. The potential categories may vary, including for example, all actions that can be performed with a syringe and hands or all actions that can be performed with gloved/ungloved hands. In other examples, the categories may be grouped by action, including all tools that can be held, rotated, scrubbed, and so on.

Clinical step definition engine 114 is also configured to store the set of rules with clinical step data store 132. Clinical step data store 132 may be subsequently accessed during the inference phases of each trained machine learning model (e.g., first machine learning model engine 116 and clinical procedure machine learning model engine 118). The use of the rules may help the system determine whether the clinical step is satisfied. For example, when the objects and actions that are detected in the digital file are matched to rules/thresholds in clinical step data store 132, the clinical step may be completed to a clinically appropriate level.

First machine learning model engine 116 is configured to process a digital file with a machine learning model. For example, the machine learning model (e.g., a foundational model) may detect patterns and objects in digital files. The machine learning model may be fine-tuned to relate the patterns/objects in the digital file to actions/objects for clinical steps. A second machine learning model (e.g., clinical procedure machine learning model engine 118) may be trained to determine whether the clinical steps are conducted to a clinically appropriate level, as further discussed herein. The output from these models may generate a set of values, including a completion indicator that identifies whether the set of rules for the clinical step are satisfied or the differences between the clinical steps and the actions. The feedback may be provided to a feedback device associated with a completion indicator.

First machine learning model engine 116 may correspond to various types of models, including a foundational model. The foundational model may be trained to identify patterns, connections, and structures in data and may be implemented in communication with a second machine learning model or fine-tuning procedure of the foundational model (e.g., via clinical procedure machine learning model engine 118). The first machine learning model may be considered foundational based on the model's ability to be modified or fine-tuned for tasks or domains with minimal additional training. The model may be trained to recognize and categorize objects or concepts absent receiving data associated with those categories or concepts beforehand. In some examples, first machine learning model engine 116 can generate output as a prediction of what the features in the digital file represent based on the training process, like a person or object/tool.

In some examples, first machine learning model engine 116 may receive an existing machine learning model from an external source, illustrated as existing machine learning model 140. Existing machine learning model 140 may correspond to a foundational model that is trained on vast datasets so it can be applied across a wide range of use cases. Illustrative examples of the second machine learning model may include, for example, Lag-LLAMA (e.g., an open source time series foundational model). In some examples, existing machine learning model 140 is trained to detect objects in an image and provide a classification of the object as output. The output from first machine learning model engine 116 or existing machine learning model 140 may be used to detect objects in the digital file before the actions or clinical steps in the digital file are detected.

In some examples, first machine learning model engine 116 may detect a tool/object in the digital file. The detected object may be identified by comparing the object to a set of known objects in an object library. First machine learning model engine 116 may receive the digital file as input, compare the detected object with the set of known objects, and provide a confidence score associated with the likelihood that a particular object was detected. The object with the highest score may be selected to be the detected object.

In response to detecting the particular object, first machine learning model engine 116 may also determine characteristics of the tool/object in the digital file. For example, the detected object may be identified for a duration/length of time in the digital file, the duration/length of time that the detected object was operated by the user, the connections between the object and other objects, and other information discussed throughout the disclosure.

In some examples, first machine learning model engine 116 utilizes two models that are trained for different purposes and used together to detect the objects/actions in the digital file. For example, a first machine learning model is trained to detect the patterns and objects in digital files (e.g., the foundational model) and a secondmachine learning model correlates the detected patterns and objects from the first machine learning model to actions, timing, positioning, and the like.

Clinical procedure machine learning model engine 118 is configured to determine whether the detected object/action identified in the digital file correspond to rules defined in clinical steps. For example, during a training phase and before the clinical procedure machine learning model has been operationalized in a production environment, the clinical procedure machine learning model may learn associations between objects/actions (e.g., by first machine learning model engine 116) and clinical steps (e.g., stored in clinical step data store 132 by clinical step definition engine 114). In some examples, a bounding box is placed around the detected object and a rule is identified as being satisfied (e.g., in accordance with the defined threshold). In other examples, detection is binary (e.g., the rule is or is not met). When the rule is associated with timing, positioning, or other defined numerical values, the training process may be associated with a delta value that compares the detected value in the digital file with the determined value in the rule. The training can set limits of when the rule is met. The clinical procedure machine learning model can learn the association between the image and the identification that the rule has been satisfied during the training process.

During an inference phase, when the clinical procedure machine learning model is operational/in use, the digital file (corresponding to the recording of the actions) may be provided as input to the model. The clinical procedure machine learning model may detect and classify objects/actions in the digital file and generate an output that includes a completion score. The completion score may correspond to the likelihood that the object/action in the digital file satisfies the rule defined for the clinical step. When the completion score exceeds a threshold, the object/action may be confirmed to satisfy the rule and the clinical step is performed to the clinically appropriate level in the clinical setting.

As an illustrative example, a clinical procedure may require performing a first clinical step for fifteen seconds followed by a second clinical step of positioning a tool at a 45-degree angle. Clinical procedure machine learning model engine 118 can detect the actions in the digital file, including the duration of actions and orientation of thetools/objects, and determine whether the actions are detected for the appropriate duration, orientation, angle, and other defined rules for the clinical step(s). When the rules are met for the clinical step (e.g., 100% or greater) or within a threshold range for the clinical step (e.g., between two values), a completion score may be determined. In other examples, the clinical procedure machine learning model can identify when an activity is initiated (e.g., 0% as a completion score), whether objects/tools identified in the digital file are being used correctly, whether the action is conducted for a duration of time to a clinically appropriate level (e.g., 100% as a completion score to complete the clinical step), and other determinations in view of the set of rules for the clinical steps, as discussed throughout the disclosure.

Clinical procedure machine learning model engine 118 is configured to receive feedback and rerun a subsequent training phase. The weights/biases of the model that correspond to detected actions, tools, and objects may adjusted, which can affect whether the rules defined in clinical steps are met or not met. With the feedback, the clinical procedure machine learning model may learn the updated associations between objects/actions (e.g., by first machine learning model engine 116) and clinical steps (e.g., stored in clinical step data store 132 by clinical step definition engine 114). The updated model may be used during the inference phase to process the digital file with a machine learning model configured to process the digital file and generate the completion indicator.

In some examples, first machine learning model engine 116 may generate output as a feature vector (e.g., information about objects in a digital file, including color, shape, and pixel values). The feature vector may be provided an input to clinical procedure machine learning model engine 118, which initiates a subsequent analysis process on the generated feature vector from the first model. For example, the feature vector may be compared to a set of defined objects/tools of words or other lookup table (LUT) in the vascular care space. The LUT may comprise the rules relating to the clinical steps of a clinical procedure that can be performed to a clinically appropriate level (e.g., medication administration, constructing connectors or other devices, line care, catheter placement, and other forms of vascular access management). The recognizedobjects/tools/actions by clinical procedure machine learning model engine 118 may be provided as the feedback (via feedback engine 120).

Feedback engine 120 is configured to generate a set of values with a completion indicator. For example, using clinical procedure machine learning model engine 118, the system may identify when an action associated with a clinical step is initiated, whether objects/tools are being used correctly, which objects/tools are detected and connected to each other, whether the action is conducted for a duration of time to a clinically appropriate level, a length of time that the action was performed or other numerical values (e.g., in association with completing an action), and other features discussed throughout the disclosure. The set of values may also comprise metrics associated with the detected actions, including initiation, duration, completion, or other values.

The set of values may also comprise a completion indicator. The completion indicator may illustrate whether the action is performed at the clinically appropriate level to satisfy the rules of the clinical step. For example, feedback engine 120 may compare the rules stored in clinical step data store 132 with processing performed by clinical procedure machine learning model engine 118. The difference/delta between the rules and the identified action may correspond with ranges of the completion indicator. The completion indicator can be provided as text, images, sound, or other mediums (e.g., highlighting a portion of the set of values in green, providing haptic feedback for a portion of the set of values for reaching the threshold, providing an audible signal, etc.).

The completion indicator may be adjusted based on a threshold value. For example, the completion indicator may comprise a first color or audible signal for reaching a first threshold value (e.g., 100%) and may comprise a second color or audible signal for being between a threshold range (e.g., 25%-75%). The adjustment to the completion indicator may include blinking or flashing (in visual feedback), increasing the volume (in audible feedback), or increasing a rate/intensity (in haptic feedback), as some illustrative examples that do not divert from the essence of the disclosure. In some examples, the clinical steps that are detected in the digital file may be used to generate feedback aboutcompletion of the overall clinical procedure (e.g., 100%). Various other colors, sounds, or haptics may be provided as feedback.

The feedback may be adjusted based other values/factors as well. For example, a set of rules may define an action as corresponding to dangerous behavior and the inference phase of the clinical procedure machine learning module may determine the correlation between the action and the rules exceeds a threshold value (e.g., within 90% of the comparison between the clinical step and the detected action). The feedback for the dangerous behavior may comprise a third color or audible signal (e.g., red color or loud warning signal). In some examples, feedback engine 120 may compare the identified clinical step in real-time and the comparison with each of the determined thresholds may be used to adjust the feedback to identify the dangerous behavior.

The feedback may comprise other values as well. For example, the set of values (e.g., actions performed, duration of the actions, tools/connections detected, etc.) may be added to a feedback document during an automatic documentation process of the clinical actions. In some examples, the feedback document may correspond to a patient's electronic medical record.

The feedback may be provided through various means. For example, the feedback may be provided visually to a user interface (e.g., dashboard), through haptic feedback (e.g., buzzing or vibrating), through audio/visual feedback, or other real-time feedback that may be provided while the action is being conducted. In some examples, the feedback may be provided as asynchronous feedback to a distributed system (e.g., to a patient's electronic medical record). The timing of the feedback may assist with a clinical training process and allow the user to make real-time adjustments to actions in response to the feedback. In some examples, the feedback may identify whether the threshold is reached and provided as a completion indicator (e.g., highlighting a value green, providing haptic feedback of reaching the threshold, etc.). Additional thresholds may be stored as well (e.g., 50% to a clinically appropriate level is yellow feedback, etc.). Using the feedback, the clinician can adjust the actions being performed to more closely align to the rules of the clinical step.

Feedback device 150 is configured to receive the feedback from compute component 100 (e.g., generated by feedback engine 120) and provide the feedback to a clinician user. The feedback may comprise, for example, visual feedback. Visual feedback may include updates to a user interface (e.g., via a display incorporated with a user device or monitor). In some examples, the feedback may comprise audio feedback (e.g., via a speaker). In some examples, the feedback may comprise haptic feedback, including kinesthetic feedback (e.g., active and resistive force feedback) or tactile feedback (e.g., vibration, texture, and heat).

In some examples, feedback device 150 utilizes a display, such as a liquid crystal display (LCD) (or touch screen), for displaying the feedback to the clinician user. Feedback device 150 may include a user interface module to implement a GUI that may be stored in a mass storage device as executable software codes that are executed by a processor of feedback device 150, as discussed herein.

In some examples, feedback device 150 generates an audible signal that provides a secondary confirmation that an action is complete. In some examples, feedback device 150 utilizes a speaker to provide audible feedback or the secondary confirmation. For example, the speaker may emit an audible signal that is stored with clinical step data store 132 to provide feedback. In the instance where a secondary confirmation is generated, the audible signal may identify that the action corresponds to clinical steps that are conducted to a clinically appropriate level (e.g., an utterance, beep, etc.).

In some examples, feedback device 150 receives an audible signal or utterance from a user or second feedback device that provides a confirmation that an action is complete. Feedback device 150 may utilize a microphone to receive the audible signal and provide the audible signal to an automatic speech recognition (ASR) process. The ASR process may be incorporated with feedback device 150 or provided as a service from a third party device. The feedback can be a suggestion that the action is complete, subject to the secondary confirmation from the second feedback device or user (e.g., utterance).

The ASR process may implement a hidden Markov model (HMM) or similar process to perform speech recognition. For example, the device implementing ASR may receive an audible signal and partition the audible signal into overlapping or windowed frames. The device may apply a fast Fourier transform (FFT) to each frame to convert it to the spectral domain and obtain the spectral energy density in each frame. The device may apply a Mel scale transformation to the frequency domain and extract the Mel Frequency Cepstral Coefficients (MFCC) and other values using a Discrete Cosine Transform (DCT). The sequence of MFCC feature vectors for the frames may be compared to each of a set of HMMs corresponding to a vocabulary of words or other lookup table (LUT). The recognized words may be provided as input to a machine learning model, where the output is the probability of observing a detected word (e.g., the highest score corresponds to the detected word). When the detected word corresponds to a known secondary confirmation that the action is complete, the detected utterance may be provided as feedback to compute component 100 (e.g., generated by feedback engine 120) or to a clinician user.

In some examples, feedback device 150 may utilize an actuator. For example, the actuator may include an electromagnetic actuator such as an Eccentric Rotating Mass (“ERM”) in which an eccentric mass is moved by a motor, a Linear Resonant Actuator (“LRA”) in which a mass attached to a spring is driven back and forth, or a “smart material” such as piezoelectric, electroactive polymers or shape memory alloys. In some examples, feedback device 150 may include electrostatic friction (“ESF”) devices or ultrasonic surface friction (“USF”) devices, or devices that induce acoustic radiation pressure with an ultrasonic haptic transducer. Other devices use a haptic substrate and a flexible or deformable surface, and devices can provide the haptic output can initiate a puff of air using an air jet incorporated with the device.

In some examples, feedback device 150 may be activated or deactivated in association with the completion indicator. For example, compute component 100 may transmit a command to feedback device 150 if the action associated with the clinical step is not complete. Feedback device 150 may process the command and cause feedback device 150 to pause or prevent operation. The activation/deactivation may be initiated fora determined amount of time and toggled in response to the command (e.g., via a lookup table or stored rules, etc.).

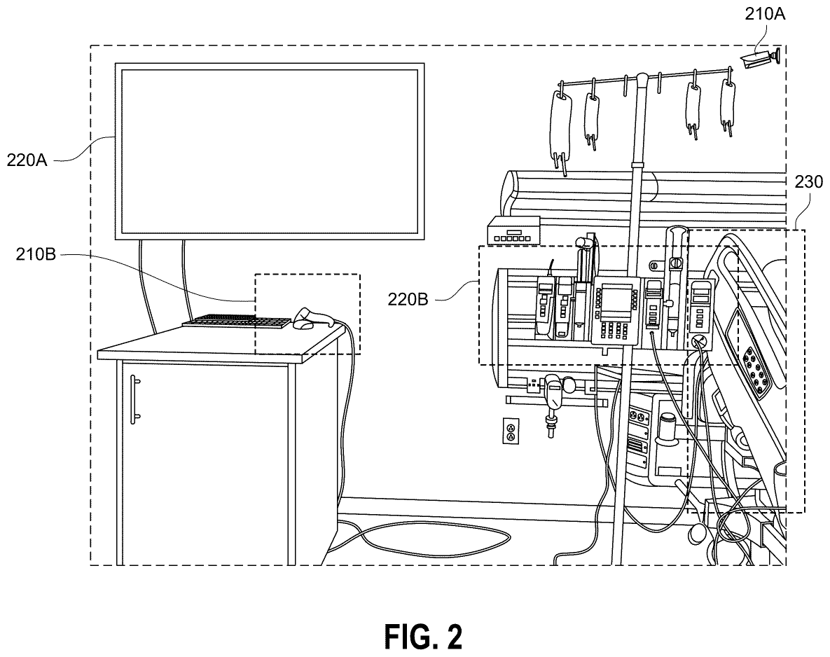

FIG. 2 illustrates an environment that comprises a sensor and feedback device, in accordance with some examples of the disclosure. In this illustration, a clinical procedure may be performed in the clinical environment. The actions associated with the clinical procedure may be captured and stored by one or more sensors 210 in the environment, including by first sensor 210A that is located above hospital bed 230 and second sensor 210B that is movable to detect actions associated with the clinical environment.

The clinical procedure may comprise a set of clinical steps, which can vary by implementation. As an illustrative example, the clinical procedure is a process to convert an intravenous (IV) fluid infusion to a saline lock and remove the peripheral IV. The clinical steps in the clinical procedure may include, for example, performing hand hygiene, collecting the supplies, stopping IV infusion with a clamp or turn off the EID, applying clean gloves, scrubbing the connection area between the hub and IV tubing for 15 seconds, letting the area dry for 30 seconds, disconnecting the primary tubing from the extension tubing, confirming that the positive pressure cap remains on the extension tubing, placing a sterile cap on end of IV tubing, scrubbing the hub for 15 seconds, letting the area dry for 30 seconds, attaching a 10 ml syringe prefilled with saline, flushing the saline lock to clear the positive pressure cap, removing syringe and discard, clamping the extension tubing, wiping top of positive pressure cap with alcohol swab to remove fluid residue, and removing the gloves.

The clinician may perform a set of actions in association with the clinical procedure at hospital bed 230, which are recorded in a digital file by first sensor 210A or second sensor 210B. The digital file may be received by a compute component (not shown) and processed with machine learning model(s). The model(s) are configured to identify the objects/tools in the digital file and correlate the actions performed by the clinician with clinical steps of the clinical procedure.

In this example, the machine learning model may identify the differences between the clinical steps and the actions in real-time. For example, the clinician mayinitiate an action to scrub the connection area between the hub and IV tubing for 15 seconds and let the area dry for 30 seconds, as actions that correspond to two clinical steps. The digital file may record the clinician initiating the scrubbing process at the connection area, in which feedback is generated and provided to the feedback device with respect to the first clinical step. The completion indicator may be provided as feedback to user interface 220 in real-time as the actions are being performed at hospital bed 230.

In this example, the clinician may view or hear the feedback as the action is being performed and be able to adjust the actions to comply with the required clinical steps of the clinical procedure based on the feedback. For example, when the actions are not started or otherwise incomplete, the feedback may identify a first color (e.g., red or yellow, respectively) as the completion indicator. When the actions are complete as defined in the clinical steps for the clinical procedure, the feedback may identify a second color (e.g., green) as the completion indicator. When the action is identified by the machine learning model as corresponding to the second clinical step, the last completion indicator for the first clinical step may remain on the user interface and the completion indicator associated with the second clinical step may be updated on the user interface.

In this example, the feedback device may be shown at various user interfaces 220, illustrated as first user interface 220A and second user interface 220B. The feedback may be provided via user interfaces 220 as the completion indicator(s). The clinician user may view the visual feedback (e.g., updates to a user interface) or audio feedback (e.g., via a speaker) as they are performing the actions in real-time.

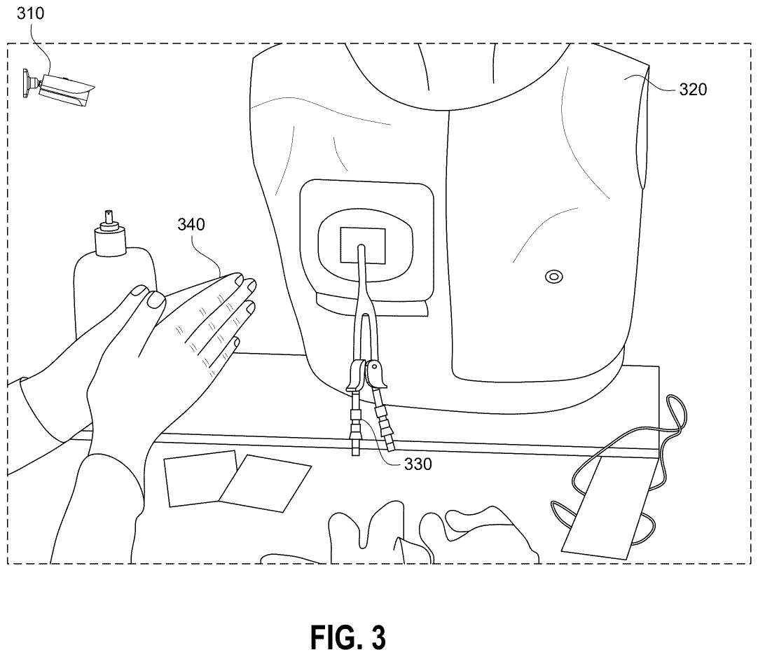

FIG. 3 illustrates an environment that comprises a sensor, in accordance with some examples of the disclosure. In this illustration, a clinical procedure may be performed in the clinical environment. The actions associated with the clinical procedure may be captured by sensor 310 that is located above a patient 320. The clinical procedure in this example may be associated with a medical technique that administers fluids, medications, and nutrients directly into a vein of patient 320 through an intravenous line 330 or cannula. The clinical steps associated with the clinical procedure may include similar clinical steps as described in FIG. 2.

An illustrative clinical step may correspond to performing hand hygiene. The clinician may initiate an action 340 that is captured by sensor 310 to generate a digital file. The digital file may be received by a first machine learning model to determine the existence of the hands (e.g., by the first machine learning model or foundational model that detects objects in an image) or actions (e.g., the hands of the clinician performing hand hygiene). The detected actions/objects from the first machine learning model may be provided as input, with the digital file, to the clinical procedure machine learning model. The clinical procedure machine learning model can determine whether the set of rules for the clinical step are satisfied and identify the differences between the clinical steps and the actions in real-time. The system may generate feedback based on the comparison. The feedback may be provided to a feedback device (not shown) with a completion indicator. The clinician may receive the feedback as the action is being performed and be able to adjust the actions to comply with the required clinical steps of the clinical procedure based on the feedback.

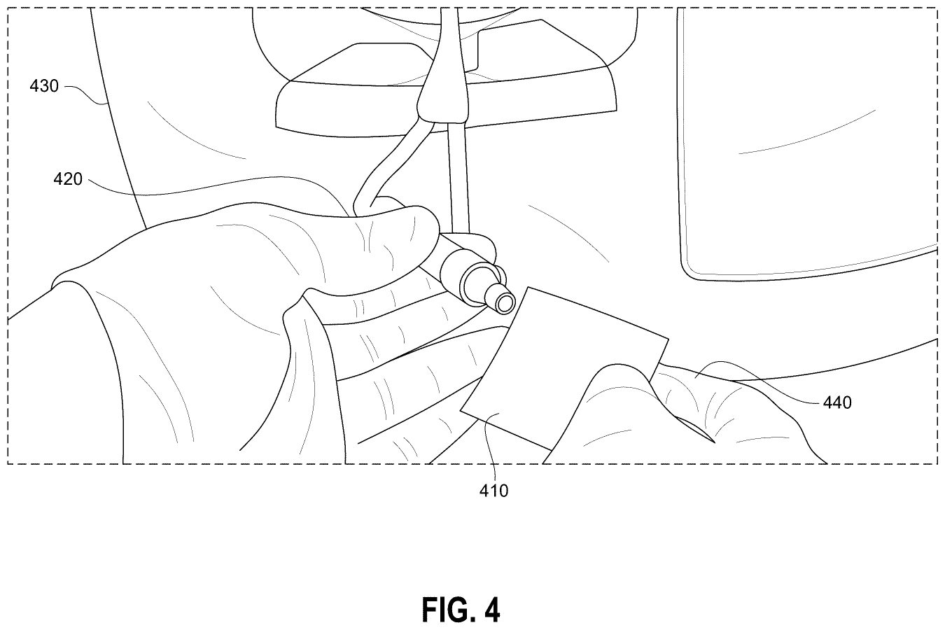

FIG. 4 illustrates an environment that comprises a sensor, in accordance with some examples of the disclosure. In this illustration, the clinical steps associated with the clinical procedure may include scrubbing the connection area between the hub and IV tubing for 15 seconds and letting the area dry for 30 seconds. The objects associated with the clinical steps may include alcohol swab 410 and IV tubing 420 associated with patient 430. The actions performed with these objects may include moving the clinician's hands 440 in a rotating motion for 15 seconds while holding alcohol swab 410.

Each of these actions and objects may be captured in the digital file that is received by the first machine learning model. The clinical procedure machine learning model may process the file to identify the differences between the rules defining the clinical steps and the actions in real-time. The system may then generate feedback. The feedback may be provided to a feedback device (not shown) with a completion indicator. The clinician may receive the feedback as the action is being performed and be able to adjust the actions to comply with the rules of the clinical steps based on the feedback.

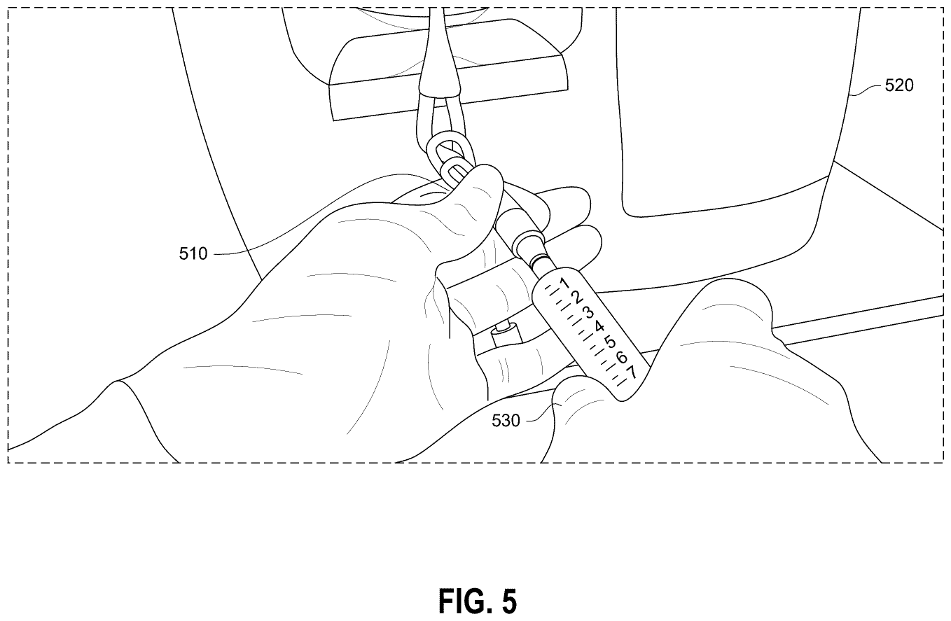

FIG. 5 illustrates an environment that comprises a sensor, in accordance with some examples of the disclosure. In this illustration, the clinical steps associated withthe clinical procedure may include attaching a 10 ml syringe prefilled with saline. The objects associated with the clinical steps may include IV tubing 510 associated with patient 520. The actions performed with these objects may include moving the clinician's hands 530 to push the IV tubing onto the syringe, where both the IV tubing and the syringe are identifiable objects in the image.

Each of these actions and objects may be captured in the digital file that is received by the machine learning model. The machine learning model may identify the differences between the clinical steps and the actions in real-time and generate feedback. The feedback may be provided to a feedback device (not shown) with a completion indicator. The clinician may receive the feedback as the action is being performed and be able to adjust the actions to comply with the required clinical steps of the clinical procedure based on the feedback.

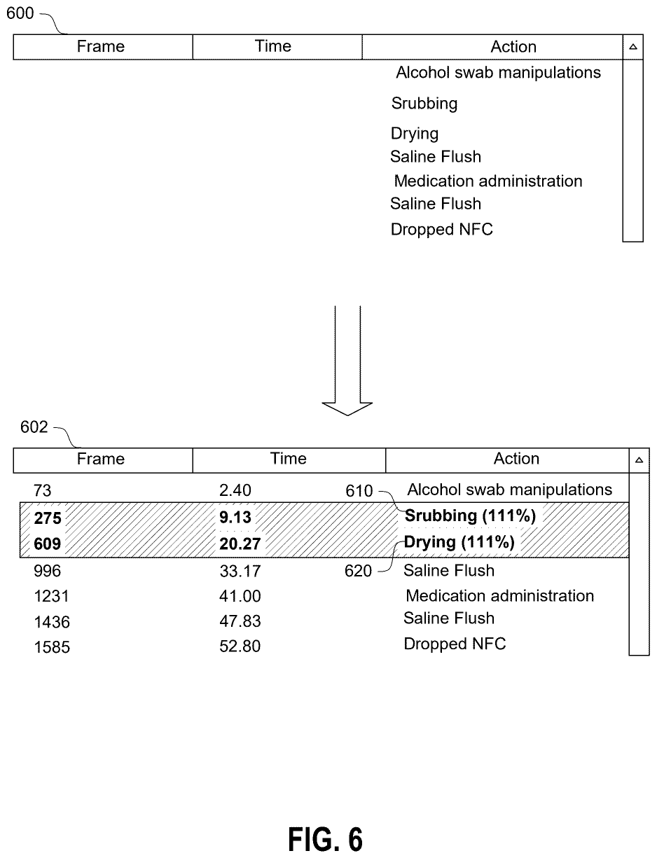

FIG. 6 illustrates feedback associated with the image detection, in accordance with some examples of the disclosure. In this example, a set of clinical steps of a clinical procedure is provided via a graphical user interface 600 at a feedback device. The clinical steps include alcohol swab manipulations, scrubbing, drying, saline flush, medication administration, saline flush, and dropped NFC.

In response to receiving a digital file representing actions performed in a clinical environment, the actions and object in the digital file may be detected by a first machine learning model. The clinical procedure machine learning model may compare the rules of the clinical steps to the detected actions/objects. As the rules are met, the completion indicator may be activated at graphical user interface 602 at a later time. In this example, two completion indicators 610, 620 are activated for two clinical steps, including a first completion indicator 610 for scrubbing and a second completion indicator 620 for drying.

Other values of may be generated by the machine learning model as well, including an indication of the image where the action was detected in the digital file (e.g., 275 for scrubbing and 609 for drying), a time duration corresponding to the length that the action lasted (e.g., 9.13 seconds for scrubbing and 20.27 seconds for drying), and a percentage completion (e.g., 111% for scrubbing and 111% for drying). These valuesmay be provided as feedback with a completion indicator that identifies that clinical steps are conducted to a clinically appropriate level (e.g., highlighted in green, buzzing, etc.). The clinical steps that did not satisfy a clinically appropriate level of completion may be provided without the completion indicator or with an alternate completion indicator (e.g., yellow/red, highlighted in gray, etc.).

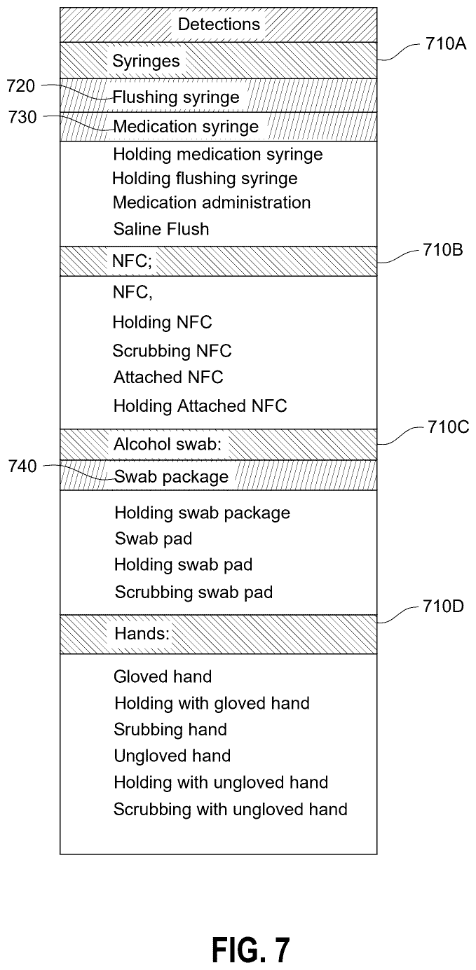

FIG. 7 illustrates feedback associated with the image detection, in accordance with some examples of the disclosure. In this example, a graphical user interface at a feedback device provides rules of clinical steps relating to groups of objects 710. For example, a first group 710A related to a syringe object may include flushing a syringe, medication with a syringe, holding a medication syringe, holding and flushing a syringe, and a saline flush. In another example, a second group 710B related to a needle-free connector (NFC) object may include NFC, holding NFC, scrubbing NFC, attaching an NFC, and holding an attached NFC. In another example, a third group 710C related to an alcohol swab object may include swab package, holding a swab package, swab pad, holding a swab pad, and scrubbing with the swab pad. In another example, a fourth group 710D related to a hands object may include gloved hands, holding an object with a gloved hand, scrubbing hands, ungloved hands, holding an object with an ungloved hand, and scrubbing an object with an ungloved hand.

In response to receiving a digital file representing actions performed in a clinical environment, the actions in the digital file may be processed during the inference phase of a first machine learning model. The clinical procedure machine learning model may compare the rules to the actions detected in the digital file. As the action is detected and the rules of the action are met, the completion indicator may be activated at the graphical user interface as the feedback. In this example, three completion indicators are activated for three clinical steps, including a first completion indicator 720 for flushing a syringe, a second completion indicator 730 for medication with a syringe, and a third completion indicator 740 for swab package.

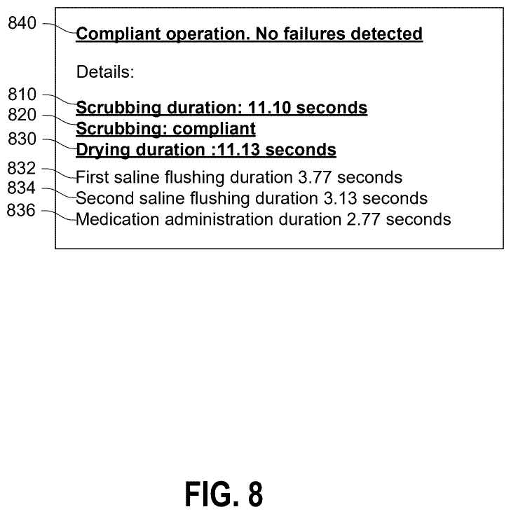

FIG. 8 illustrates feedback associated with the image detection, in accordance with some examples of the disclosure. In this example, a graphical user interface at a feedback device provides a set of clinical steps of a clinical procedure. Theclinical steps include block 810 (scrubbing duration: 11.10 seconds), block 820 (scrubbing: compliant), block 830 (drying duration: 11.13 seconds), and other actions that have not been determined to satisfy the rules of the clinical step. These other actions include block 832 (first saline flushing duration: 3.77 seconds), block 834 (second saline flushing duration: 3.13 seconds), and block 836 (medication administration duration: 2.77 seconds).

The user interface may be updated with the feedback in real-time. For example, as the action is detected and the rules of the action are met in accordance with the clinical step, the completion indicator may be activated at the graphical user interface. In this example, three completion indicators are activated for three clinical steps, including a first completion indicator for scrubbing duration (block 810), a second completion indicator for scrubbing (block 820), and a third completion indicator for drying duration (block 830).

Other values of may be generated as feedback, including an indication that the action identified in the digital file corresponds to dangerous behavior (block 840) or failure of the clinical procedure. Here, no dangerous behavior was detected, and the feedback provided to the user interface indicates that the operation was compliant and no failures were detected.

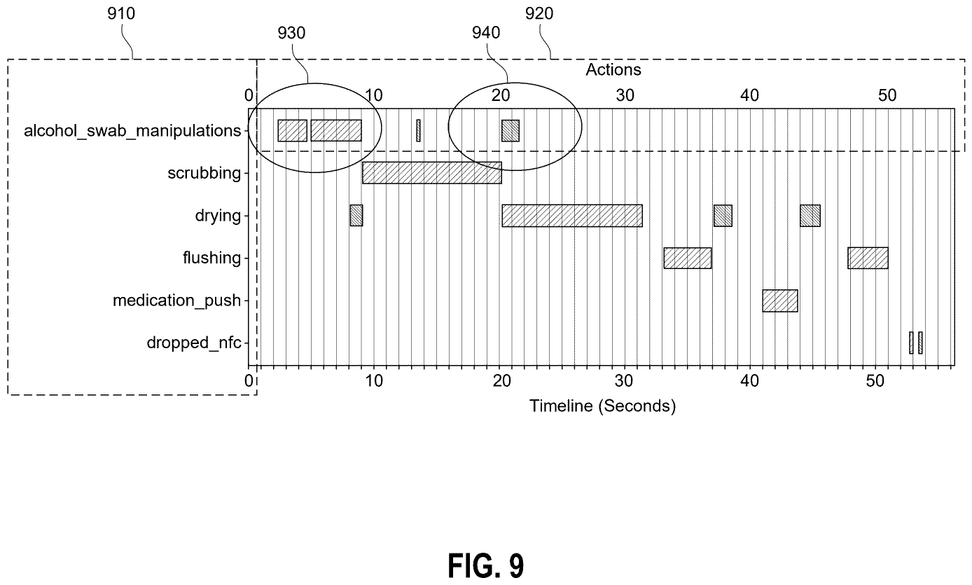

FIG. 9 illustrates feedback associated with the image detection, in accordance with some examples of the disclosure. In this example, a graphical user interface at a feedback device provides a set of clinical steps 910 with respect to a time duration 920. The clinical steps include alcohol swab manipulations, scrubbing, drying, flushing, medication push, and dropped NFC.

In some examples, the time duration 920 of the actions may be provided to the user interface as well. The actions that correspond to clinical steps that are detected in the digital file may be provided with a first completion indicator (block 930) and the actions that correspond to clinical steps that are not detected in the digital file or are detected but out of order and improper may be provided with a second completion indicator (block 940).

It should be noted that the terms “optimize,” “optimal” and the like as used herein can be used to mean making or achieving performance as effective or perfect as possible. However, as one of ordinary skill in the art reading this document will recognize, perfection cannot always be achieved. Accordingly, these terms can also encompass making or achieving performance as good or effective as possible or practical under the given circumstances, or making or achieving performance better than that which can be achieved with other settings or parameters.

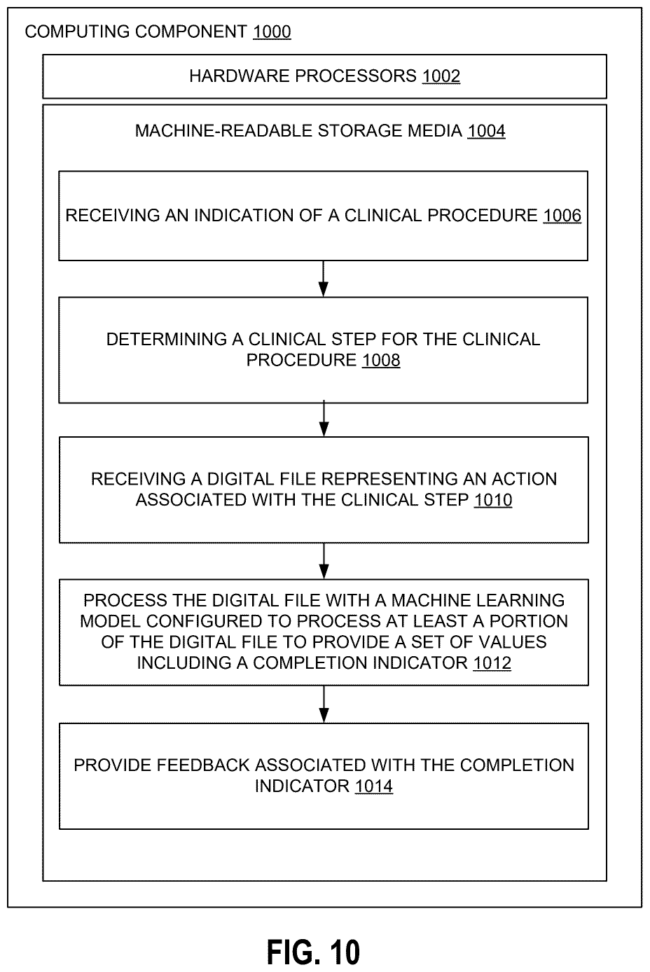

FIG. 10 is a computing component that may be used to implement examples of the disclosed technology. Computing component 1000 may be, for example, a server computer, a controller, or any other similar computing component capable of processing data. In the example implementation of FIG. 10, computing component 1000 includes hardware processor 1002 and machine-readable storage medium 1004.

Hardware processor 1002 may be one or more central processing units (CPUs), semiconductor-based microprocessors, and/or other hardware devices suitable for retrieval and execution of instructions stored in machine-readable storage medium 1004. Hardware processor 1002 may fetch, decode, and execute instructions, such as instructions 1006-1014, to control processes or operations for processes described herein. As an alternative or in addition to retrieving and executing instructions, hardware processor 1002 may include one or more electronic circuits that include electronic components for performing the functionality of one or more instructions, such as a field programmable gate array (FPGA), application specific integrated circuit (ASIC), or other electronic circuits.

A machine-readable storage medium, such as machine-readable storage medium 1004, may be any electronic, magnetic, optical, or other physical storage device that contains or stores executable instructions. Thus, machine-readable storage medium 1004 may be, for example, Random Access Memory (RAM), non-volatile RAM (NVRAM), an Electrically Erasable Programmable Read-Only Memory (EEPROM), a storage device, an optical disc, and the like. In some examples, machine-readable storage medium 413 may be a non-transitory storage medium, where the term “non-transitory” does not encompass transitory propagating signals. As described in detail below,machine-readable storage medium 1004 may be encoded with executable instructions, for example, instructions 1006-1014.

Hardware processor 1002 may execute instruction 1006 to receive an indication of a clinical procedure. For example, the indication of the clinical procedure may be received from national or local agencies or organizations. The indication may identify that information is stored in an accessible data store and available for access.

Hardware processor 1002 may execute instruction 1008 to determine a clinical step for the clinical procedure. The clinical step may be defined as a set of rules for complying with the national or local agencies or organizations. For example, the rules may include information regarding a clinical tool used, operations taken to orient the tool, a time duration of each operation, and other characteristics of the actions. The rules may define thresholds/ranges that are satisfied to complete the clinical step.

Hardware processor 1002 may execute instruction 1010 to receive a digital file representing an action associated with the clinical step. The digital file may be recorded using output from a sensor (e.g., cameras, microphones, etc.). The recording may represent an action or set of actions that is being performed by a clinician in the clinical setting. For example, the digital file may record at least one image of an action (e.g., twisting a syringe, moving a disinfectant wipe, etc.) or an object (e.g., an image of a clinician's hand, a syringe, a disinfectant wipe, etc.). The digital file may comprise a single image or set of images of the object/action.

Hardware processor 1002 may execute instruction 1012 to process the digital file with a machine learning model configured to process at least a portion of the digital file. One or more machine learning models may be implemented by the system, or may be combined to a single model without diverting from the essence of the disclosure. For example, a single machine learning model may be trained to detect patterns/objects in the digital file, relate the patterns/objects to clinical steps, and determine whether the clinical steps are conducted to a clinically appropriate level. In another example, a first machine learning model (e.g., a foundational model) may detect patterns and objects in digital files and may be fine-tuned to relate the patterns/objects in the digital file to actions/objects for clinical steps. A second machine learning model (e.g., clinicalprocedure machine learning model) may be trained to determine whether the clinical steps are conducted to a clinically appropriate level.

During an inference phase, the trained machine learning model(s) may process the digital file as input. The output of the machine learning model(s) may generate a set of values including a completion indicator. The set of values may identify when an activity is initiated (e.g., as a clinical step), whether objects/tools identified in the digital file are being used correctly, whether the action is conducted for a duration of time to a clinically appropriate level (e.g., to completion of the clinical step), and other values. The feedback may also comprise a completion indicator that illustrates/provides whether the action is performed at the clinically appropriate level to satisfy the rules of the clinical step in the clinical procedure.

Hardware processor 1002 may execute instruction 1014 to provide feedback associated with the completion indicator. For example, the feedback may be provided visually to a user interface (e.g., dashboard), through haptic feedback (e.g., buzzing or vibrating), through audio/visual feedback, or other feedback means. The timing of the feedback may assist with a clinical training process and allow the user to make real-time adjustments to actions in response to the feedback. In some examples, the feedback may identify whether the threshold is reached using a completion indicator (e.g., highlighting a value green/bold/underline, providing haptic feedback of reaching the threshold, activating/deactivating a clinical workflow, providing a numerical value, etc.). Using the feedback, the clinician can adjust the actions being performed to more closely align with the clinical step.

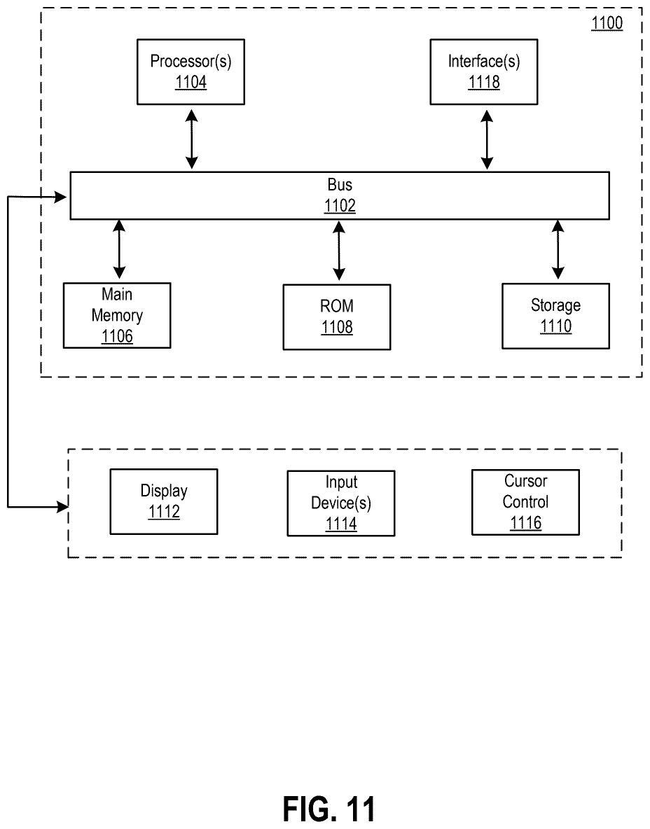

FIG. 11 depicts a block diagram of an example computer system 1100 in which various examples of the disclosed technology described herein may be implemented. The computer system 1100 includes a bus 1102 or other communication mechanism for communicating information, one or more hardware processors 1104 coupled with bus 1102 for processing information. Hardware processor(s) 1104 may be, for example, one or more general purpose microprocessors.

Computer system 1100 also includes main memory 1106, such as a random access memory (RAM), cache and/or other dynamic storage devices, coupled tobus 1102 for storing information and instructions to be executed by processor 1104. Main memory 1106 also may be used for storing temporary variables or other intermediate information during execution of instructions to be executed by processor 1104. Such instructions, when stored in storage media accessible to processor 1104, render computer system 1100 into a special-purpose machine that is customized to perform the operations specified in the instructions.

Computer system 1100 further includes read only memory (ROM) 1108 or other static storage device coupled to bus 1102 for storing static information and instructions for processor 1104. Storage device 1110, such as a magnetic disk, optical disk, or USB thumb drive (Flash drive), etc., is provided and coupled to bus 1102 for storing information and instructions.

Computer system 1100 may be coupled via bus 1102 to display 1112, such as a liquid crystal display (LCD) (or touch screen), for displaying information to a computer user. Input device 1114, including alphanumeric and other keys, is coupled to bus 1102 for communicating information and command selections to processor 1104. Another type of user input device is cursor control 1116, such as a mouse, a trackball, or cursor direction keys for communicating direction information and command selections to processor 1104 and for controlling cursor movement on display 1112. In some examples, the same direction information and command selections as cursor control may be implemented via receiving touches on a touch screen without a cursor.

Computing system 1100 may include a user interface module to implement a GUI that may be stored in a mass storage device as executable software codes that are executed by the computing device(s). This and other modules may include, by way of example, components, such as software components, object-oriented software components, class components and task components, processes, functions, attributes, procedures, subroutines, segments of program code, drivers, firmware, microcode, circuitry, data, databases, data structures, tables, arrays, and variables.

In general, the word “component,” “engine,” “system,” “database,” data store,” and the like, as used herein, can refer to logic embodied in hardware or firmware, or to a collection of software instructions, possibly having entry and exit points, written ina programming language, such as, for example, Java, C or C++. A software component may be compiled and linked into an executable program, installed in a dynamic link library, or may be written in an interpreted programming language such as, for example, BASIC, Perl, or Python. It will be appreciated that software components may be callable from other components or from themselves, and/or may be invoked in response to detected events or interrupts. Software components configured for execution on computing devices may be provided on a computer readable medium, such as a compact disc, digital video disc, flash drive, magnetic disc, or any other tangible medium, or as a digital download (and may be originally stored in a compressed or installable format that requires installation, decompression or decryption prior to execution). Such software code may be stored, partially or fully, on a memory device of the executing computing device, for execution by the computing device. Software instructions may be embedded in firmware, such as an EPROM. It will be further appreciated that hardware components may be comprised of connected logic units, such as gates and flip-flops, and/or may be comprised of programmable units, such as programmable gate arrays or processors.

Computer system 1100 may implement the techniques described herein using customized hard-wired logic, one or more ASICs or FPGAs, firmware and/or program logic which in combination with the computer system causes or programs computer system 1100 to be a special-purpose machine. According to one example of the disclosed technology, the techniques herein are performed by computer system 1100 in response to processor(s) 1104 executing one or more sequences of one or more instructions contained in main memory 1106. Such instructions may be read into main memory 1106 from another storage medium, such as storage device 1110. Execution of the sequences of instructions contained in main memory 1106 causes processor(s) 1104 to perform the process steps described herein. In alternative examples, hard-wired circuitry may be used in place of or in combination with software instructions.

The term “non-transitory media,” and similar terms, as used herein refers to any media that store data and/or instructions that cause a machine to operate in a specific fashion. Such non-transitory media may comprise non-volatile media and/or volatile media. Non-volatile media includes, for example, optical or magnetic disks, suchas storage device 1110. Volatile media includes dynamic memory, such as main memory 1106. Common forms of non-transitory media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge, and networked versions of the same.

Non-transitory media is distinct from but may be used in conjunction with transmission media. Transmission media participates in transferring information between non-transitory media. For example, transmission media includes coaxial cables, copper wire and fiber optics, including the wires that comprise bus 1102. Transmission media can also take the form of acoustic or light waves, such as those generated during radio-wave and infra-red data communications.

Computer system 1100 also includes interface 1118 coupled to bus 1102. Interface 1118 provides a two-way data communication coupling to one or more network links that are connected to one or more local networks. For example, interface 1118 may be an integrated services digital network (ISDN) card, cable modem, satellite modem, or a modem to provide a data communication connection to a corresponding type of telephone line. As another example, interface 1118 may be a local area network (LAN) card to provide a data communication connection to a compatible LAN (or WAN component to communicate with a WAN). Wireless links may also be implemented. In any such implementation, interface 1118 sends and receives electrical, electromagnetic or optical signals that carry digital data streams representing various types of information.

A network link typically provides data communication through one or more networks to other data devices. For example, a network link may provide a connection through local network to a host computer or to data equipment operated by an Internet Service Provider (ISP). The ISP in turn provides data communication services through the world wide packet data communication network now commonly referred to as the “Internet.” Local network and Internet both use electrical, electromagnetic or optical signals that carry digital data streams. The signals through the various networks and thesignals on network link and through communication interface 1118, which carry the digital data to and from computer system 1100, are example forms of transmission media.

Computer system 1100 can send messages and receive data, including program code, through the network(s), network link and interface 1118. In the Internet example, a server might transmit a requested code for an application program through the Internet, the ISP, the local network and interface 1118.

The received code may be executed by processor 1104 as it is received, and/or stored in storage device 1110, or other non-volatile storage for later execution.

Each of the processes, methods, and algorithms described in the preceding sections may be embodied in, and fully or partially automated by, code components executed by one or more computer systems or computer processors comprising computer hardware. The one or more computer systems or computer processors may also operate to support performance of the relevant operations in a “cloud computing” environment or as a “software as a service” (SaaS). The processes and algorithms may be implemented partially or wholly in application-specific circuitry. The various features and processes described above may be used independently of one another, or may be combined in various ways. Different combinations and sub-combinations are intended to fall within the scope of this disclosure, and certain method or process blocks may be omitted in some implementations. The methods and processes described herein are also not limited to any particular sequence, and the blocks or states relating thereto can be performed in other sequences that are appropriate, or may be performed in parallel, or in some other manner. Blocks or states may be added to or removed from the disclosed examples. The performance of certain of the operations or processes may be distributed among computer systems or computers processors, not only residing within a single machine, but deployed across a number of machines.

As used herein, a circuit might be implemented utilizing any form of hardware, software, or a combination thereof. For example, one or more processors, controllers, ASICs, PLAS, PALs, CPLDs, FPGAs, logical components, software routines or other mechanisms might be implemented to make up a circuit. In implementation, the various circuits described herein might be implemented as discrete circuits or thefunctions and features described can be shared in part or in total among one or more circuits. Even though various features or elements of functionality may be individually described or claimed as separate circuits, these features and functionality can be shared among one or more common circuits, and such description shall not require or imply that separate circuits are required to implement such features or functionality. Where a circuit is implemented in whole or in part using software, such software can be implemented to operate with a computing or processing system capable of carrying out the functionality described with respect thereto, such as computer system 1100.

As used herein, the term “or” may be construed in either an inclusive or exclusive sense. Moreover, the description of resources, operations, or structures in the singular shall not be read to exclude the plural. Conditional language, such as, among others, “can,” “could,” “might,” or “may,” unless specifically stated otherwise, or otherwise understood within the context as used, is generally intended to convey that certain examples include, while other examples do not include, certain features, elements and/or steps.

Terms and phrases used in this document, and variations thereof, unless otherwise expressly stated, should be construed as open ended as opposed to limiting. Adjectives such as “conventional,” “traditional,” “normal,” “standard,” “known,” and terms of similar meaning should not be construed as limiting the item described to a given time period or to an item available as of a given time, but instead should be read to encompass conventional, traditional, normal, or standard technologies that may be available or known now or at any time in the future. The presence of broadening words and phrases such as “one or more,” “at least,” “but not limited to” or other like phrases in some instances shall not be read to mean that the narrower case is intended or required in instances where such broadening phrases may be absent.

1. A system for providing computational compliance verification of vascular access management, the system comprising:
a processor; and
a non-transitory, computer-readable medium comprising instructions that when executed by the processor cause the system to:
receive an indication of a clinical procedure associated with vascular care to be performed in a clinical setting;
responsive to the indication of the clinical procedure, determine a clinical step needed for performing the clinical procedure, wherein determining the clinical step includes determining a rule that defines the clinical step;
receive a digital file representing an a monitored action, associated with vascular care, performed in the clinical setting;
process the digital file with a machine learning model configured to process at least a portion of the digital file to provide a set of values including a completion indicator, wherein processing the digital file with the machine learning model includes (i) detecting one or more objects or actions in the digital file, (ii) comparing the detected one or more objects or actions to the rule that defines the clinical step, and (iii) providing the set of values based on the comparison; and
provide feedback associated with the completion indicator.

2. The system of claim 1, further comprising:
an image sensor configured to capture the monitored action associated with the clinical step.

3. The system of claim 2, wherein the image sensor is a camera and the digital file is an image.

4. The system of claim 2, wherein the digital file is a series of images of the monitored action.

5. The system of claim 2, wherein the digital file is a stream of images of the monitored action.

6. The system of claim 1, further comprising:
a speaker, wherein the feedback associated with the completion indicator is provided through the speaker.

7. The system of claim 1, further comprising:
a microphone, wherein the feedback associated with the completion indicator is an audible signal that the action is complete based on a secondary confirmation from a feedback device or user.

8. The system of claim 1, further comprising:
a haptic device, wherein the feedback associated with the completion indicator is provided through the haptic device.

9. The system of claim 1, wherein the instructions further cause the system to:
detect whether an object is present in the digital file.

10. The system of claim 1, wherein the instructions further cause the system to:
detect whether an object is present for a duration of time in the digital file.

11. The system of claim 1, wherein the completion indicator comprises a length of time that the monitored action was performed.

12. The system of claim 1, wherein the instructions further cause the system to:
detect whether an object is present at a determined orientation that is within a threshold orientation of the clinical step.

13. The system of claim 1, wherein the processor is further configured to:
adjust the feedback to comprise the completion indicator corresponding to a dangerous behavior.

14. The system of claim 1, wherein the processor is further configured to:
in response to the monitored action not being complete, deactivate a feedback device that provides the feedback.

15. A computer-implemented method comprising:
receiving an indication of a clinical procedure to be performed in a clinical setting;
responsive to the indication of the clinical procedure, determining a clinical step needed for performing the clinical procedure, wherein determining the clinical step includes determining a rule that defines the clinical step;
receiving a digital file representing monitored action, associated with vascular care, performed in the clinical setting
providing the digital file to a trained machine learning model; configured to process at least a portion of the digital file to generate a set of values including a completion indicator; and
processing the digital file with the machine learning model, wherein the processing includes (i) detecting one or more objects or actions in the digital file, (ii) comparing the detected one or more objects or actions to the rule that defines the clinical step, and (iii) generating the set of values based on the comparison; and
providing feedback associated with the completion indicator.

16. The computer-implemented method of claim 15, further comprising:
an image sensor configured to capture the monitored action associated with the clinical step.

17. The computer-implemented method of claim 16, wherein the image sensor is a camera and the digital file is an image.

18. The computer-implemented method of claim 15, wherein the digital file is a series of images of the monitored action.

19. The computer-implemented method of claim 15, wherein the digital file is a stream of images of the monitored action.

20. A non-transitory computer-readable storage medium storing a plurality of instructions executable by a processor, wherein, when executed by the processor, the plurality of instructions cause the processor to:
in response to identifying a clinical procedure associated with vascular care to be performed in a clinical setting, determine a clinical step for the clinical procedure, wherein determining the clinical step includes determining a rule that defines the clinical step;
receive a digital file representing a monitored action, associated with vascular care, performed in the clinical setting; process the digital file with a machine learning model configured to process at least a portion of the digital file to provide a set of values including a completion indicator, wherein processing the digital file with the machine learning model includes (i) detecting one or more objects or actions in the digital file, (ii) comparing the detected one or more objects or actions to the rule that defines the clinical step, and (iii) providing the set of values based on the comparison; and
provide feedback associated with the completion indicator.
